(19) United States
(12) Patent Application Publication
(10) Pub. No.: US 2010/0196199 A1
(43) Pub. Date: Aug. 5, 2010

(54) SYSTEM AND METHOD FOR TREATMENT OF PATHOGENS IN DRIED SEWAGE SLUDGE

(75) Inventors: Armin Vonplon, Oberlunkofen (CH); Eric Liechti, Wohlen (CH)

Correspondence Address: NIXON & VANDERHYE, PC, 901 NORTH GLEBE ROAD, 11TH FLOOR, ARLINGTON, VA, 22203, US

(73) Assignee: ANDRITZ SEPARATION INC., Arlington, TX (US)

(21) Appl. No.: 12/529,483

(22) PCT Filed: Mar. 3, 2008
(86) PCT No.: PCT/US2008/055609

§ 371 Date: Apr. 6, 2010

Related U.S. Application Data
(60) Provisional application No. 60/892,403, filed on Mar. 1, 2007.

(51) Int. Cl. C02F 11/18; A61L 2/06; A61L 2/24

(52) U.S. Cl. 422/38; 422/109

(57) ABSTRACT

A method and system for treating biosolid material having pathogens, the method including: introducing dried biosolid material into a pathogen treatment vessel; monitoring an actual temperature of the dried biosolid material in the treatment vessel; determining a desired residence time of the biosolid material in the treatment vessel; determining a desired minimum temperature of the dried biosolid material in the treatment vessel, wherein the minimum temperature is based on the desired determined residence time of the biosolid material in the treatment vessel; passing the dried biosolid material through the treatment vessel such that the actual residence time of the biosolid material in the treatment vessel is at least as long as the determined residence time; applying heat from a hot fluid to the dried biosolid material in the treatment vessel, if the actual temperature of the biosolid material is lower than the minimum temperature, and reducing pathogens in the biosolid material by maintaining the biosolid material in the treatment vessel for at least the determined residence time while the actual temperature is at least as hot as the minimum temperature.

### RELATED APPLICATION

This application claims the benefit of U.S. Provisional Patent Application Ser. No. 60/892,403 filed Mar. 1, 2007, the entirety of which is incorporated by reference.

### BACKGROUND OF THE INVENTION

The invention relates to a post drying device, particularly for sewage sludge, that treats, such as by pasteurization, pathogens of dried biosolid material, e.g., dried sewage sludge.

Dryers of various configurations, including belt, drum, fluid bed, indirect paddle dryers, indirect disc dryers have been used to dry various biosolid materials and in particular sewage sludge. Pathogens may live in the dried biosolid material. Pathogens are disease-causing organisms, such as certain bacteria, viruses and parasites. The pathogens in dried biosolids may be dangerous to humans and other animal life. Rodents and insects may pick up pathogens from the dried biosolids in waste treatment facilities and carry the pathogens to humans and other animals. There is a long felt need for systems and methods to treat pathogens, e.g., destroy the pathogens, in dried biosolids before they can be carried out of the waste treatment facility and create health risks to humans and animals.

In 1993, the U.S. Environmental Protection Agency (EPA) promulgated rules to ensure public safety in the treatment and handling of sewage sludge. In particular, the EPA issued rules know as the “Part 503 Rule” and described in EPA publications EPA/625/R-92/013 (December 1992) and entitled “Control of Pathogens and Vector Attractions in Sewage Sludge”; EPA/832-B-92-005 entitled “Domestic Septage Regulatory Guidance: A Guide to the EPA 503 Rule,” and EPA/832/R-93/003 (September 1994) entitled “A Plain English Guide to the EPA Part 503 Biosolids Rule.” Particularly, Chapter 5 of EPA/832/R-93/003 entitled “Pathogen and Vector Attraction Reduction Requirements” to address treatment of biosolids to address pathogens.

The EPA Part 503 Rule sets forth treatment regimens to ensure that pathogens remain at or below acceptable levels in biosolids. See EPA/832/R-93/003 (Chapter 5). The treatment regimens include temperature and residence time requirements for the treatment of biosolids. For example, biosolids in the form of small particles are to be heated by contact with a warm gas or an immiscible liquid. Particularly, biosolids having a composition of seven percent (7%) solids or greater, e.g., dried biosolids, are to be subjected to a temperature of at least 50 degrees Celsius (50° C.) for a residence time of at least 15 seconds (sec.). See Regime B in Table 5-3 of EPA/832/R-93/003 (Chapter 5, p. 112).

To satisfy the temperature and residence time requirements, the EPA published time-temperature relationship equations to assist operators of waste treatment facilities to satisfy at least some of the requirements of the Part 503 Rule. As an example, the time-temperature relationship equation published by the EPA for the Part 503 Rule is:

$$D \geq \frac{131 \text{,} 700 \text{,} 000}{{10}^{0.14 t}}$$

In this relationship equation, “D” represents the days of treatment of the biosolid needed to comply with the Part 503 Rule, and “t” represents the temperature in degrees Celsius of the treatment.

To comply with the EPA Part 503 Rule, waste treatment operators must determine the residence time (D) for the solids and monitor the temperature of the biosolids during the required residence time. There are difficulties involved in monitoring and maintaining the temperature of the biosolids in a continuous waste treatment system. In view of these difficulties, there is a long felt need for systems and methods for treating dried particulate biosolids in a continuous waste treatment system to minimize pathogens in the biosolids and comply with the EPA Part 503 Rule.

### SUMMARY OF THE INVENTION

The system and methods described herein treat particulate biosolids such that the temperature and residence time of the biosolids in a pathogen treatment vessel are monitored and controlled. The system and method may control the biosolids to minimize pathogens and ensure compliance with the EPA Part 503 Rule.

A holding vessel has been developed having a known volume or known material volume receives dried particulate biosolids, such as a continuous feed of biosolids to the top of the vessel. The holding vessel comprises: a discharge valve of regulating the flow rate of the biosolids through the vessel to control the residence time of the biosolids in the vessel; a hot gas supply to heat the biosolids in the vessel by a hot gas that percolates through the biosolids in the vessel; a controller regulates the flow of hot gas percolating through the vessel, and regulating the flow rate of the biosolids through the vessel using as a feed back the temperature signals from one or more temperature sensors in the vessel monitoring the temperature of the biosolids.

A controller has been developed to monitor the temperature and residence time in a continuous flow tank, and determine if the temperature and residence time meet or exceed a prescribed time-temperature relationship, such as one or more of the relationships set forth in EPA Part 503 Rule. To ensure that the residence time of biosolids in the tank is at least for a period determined by the controller, the controller may adjust the rotational speed of a tank discharge valve, where the valve has a known volumetric throughput based on speed of rotation. To ensure that the temperature of the biosolids in the tank is at least at a temperature determined by the controller, the controller may compare one or more measured temperatures of the biosolids in the tank to a desired temperature and adjust a flow of a heated fluid bubbling through the tank to add heat to the biosolids if the measured temperatures are below the desired temperature.

A method has been developed for treating dried biosolid material having pathogens, the method comprises: introducing the dried biosolid material into a pathogen treatment continuous flow vessel; monitoring an actual temperature of the biosolid material as the material flows through the vessel; determining a residence time of the biosolid material in the vessel and determining a desired minimum temperature of the biosolid material in the vessel, wherein the desired minimum temperature is based on a determined residence time of the biosolid material in the vessel; adjusting a flow rate of the biosolid material through the vessel to ensure an actual residence time of the biosolid material in the vessel is at least as long as the determined residence time; percolating a hot fluid through the biosolid material in the vessel, if the actual temperature of the biosolid material is lower than the desired minimum temperature, and reducing pathogens in the biosolid material by maintaining the biosolid material in the vessel for at least the determined residence time while the actual temperature is at least as hot as the desired minimum temperature.

In the method, the biosolid material may be a particulate material, such as dried sewage sludge. The biosolid material may be discharged from a drum dryer or belt dryer before being introduced into the pathogen treatment vessel. Further, the biosolid material may be introduced at a continuous rate into the vessel. In addition, the hot fluid may be a hot gas, such as hot air from a dryer used to dry the biosolid material before the material enters the vessel.

### BRIEF DESCRIPTION OF THE DRAWINGS

The invention will now be described using the examples in the drawings where:

FIG. 1 is a schematic diagram of a drying plant having a drum dryer and a treatment system for treating pathogens in biosolids.

FIG. 2 is a schematic diagram of a drying plant having a belt dryer and a treatment system for treating pathogens in biosolids.

### DESCRIPTION OF THE PREFERRED EMBODIMENTS

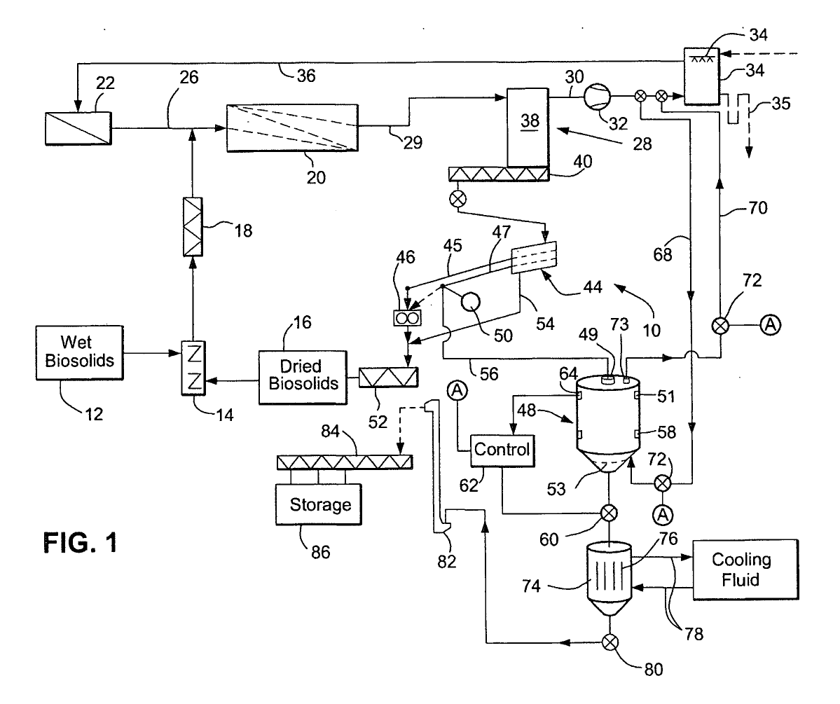

FIG. 1 is a schematic diagram showing a drying plant 10 suitable for drying biosolid materials, such as sewage sludge. Wet biosolids are provided from a storage silo 12 to a mixer 14 that also receives previously dried recycled biosolid materials 16. The mixer 14 may be controlled to maintain a desired moisture content of the mixture of wet and recycled dried biosolids discharged from the mixer and to prevent agglomeration of the mixture.

Moisture sensors in the mixer, storage silo and a container for the previously dried materials 16 may be used to provide feedback data to regulate the input flows from the silo 12 and of sludge to maintain the desired moisture of sludge in the mixer 14. The ratio of wet biosolid material from silo 14 mixed with dried recycled biosolid material 16 is adjusted to achieve a desired moisture content of the material discharged from the mixer mixture.

A screw conveyor 18 feeds the biosolids mixture to a dryer drum 20 where the biosolids mixture is dried by hot air fed through conduit 26. The biosolids mixture may or may not be conveyed to the dryer drum 20 through conduit 26. Drying air is heated in a heat exchanger 22, passes over the biosolid material in the dryer 20 and is exhausted from the drying drum through exhaust conduit 29 that may be pipe or other conduit that conveys hot gases and sludge.

The dried material and the drying air are carried through conduit(s) 29 to a filter plant 28 where the drying air is extracted from the dried biosolids material. The extracted drying air flows through line 30 to a conveying fan 32 and then to a condenser 34 where water vapor in the drying air is condensed and extracted through a drain line 35. Gas collectors, such as air apertures 37, collect the waterless drying air from the condensor. The extracted drying air is carried through a line 36 to the heat exchanger 22 for reheating and feeding to the drying drum 24 thereby forming a closed drier loop for dry air.

The filter plant 28 removes the dried biosolid material from the drying air, stores the dried biosolids in a storage silo 38 and conveys the dried material by a conveyor 40 to a screen separator 44. The screen separator 44 separates the coarse biosolid particles and conveys 45 the coarse particles to a grinder 46 for reducing the size of the particles. Medium size particles are also recovered from the filter screen 44 and conveyed 47 to a grinder 46 or to a pathogen treatment system 48. Small size particles separated from the screen separator are conveyed 54 for mixing with the ground dried material in conveyor 52. The small dried particulate biosolids and the particulate biosolids from the grinder 46 may be conveyed via screw conveyor 52 to the dried biosolids storage tank 16.

Medium sized biosolid particles transported on conveyor 47 are, for example, particles sized to be treated under Regime B for Class A Pathogen Reduction Under Alternative 1 (See Table 5-3 of EPA/832/R-93/003 (Chapter 5, p. 112)). A biosolid router device 50 may determine the path of medium size particles, and may include, a controllable blade directing biosolid material to the grinder 46 or conveyor 56. The router 50 determines the portion of the dried biosolids to be directed to the grinder 46 and the portion directed to be to the treatment vessel 48 via conveyor 56.

The dried biosolids may enter the treatment vessel 48, which may be a silo, tank or other closed vessel, at the top of the vessel 49. The flow of biosolids into the vessel 48 may be a continuous flow. The dried biosolid particles move through the vessel 48 and are discharged through the bottom of the vessel and to a valve 60 which regulates the flow rate of the biosolid particles. The valve 60 may be inside the vessel or at a discharge outlet that is external to the vessel.

The flow rate of biosolids particles through the valve determines the minimum residence time of the particles in the vessel 48, especially if a continuous flow of biosolids enters the vessel and the upper surface of particles in the vessel remains relatively constant in the vessel. A level sensor 51 monitors the level of biosolids in the vessel 48. If the flow of biosolids is not continuous, the valve 60 may be closed to ensure that the biosolids in the vessel have at least a sufficient resident time to minimize pathogens in the biosoils. The vessel is closed for at least the reason to reduce the loss of heat from the biosolids in the vessel. The pressure in the vessel may be at or near atmospheric pressure.

Preferably a control system 62, e.g. a computer control system, monitors the flow of biosolids into the vessel 48, the level sensor and the rate of particles discharged from the vessel to ensure that the residence time of the biosolids is no shorter than a desired residence time. The control system may receive outputs from flow sensors coupled to the inlet line 56 and discharge of the vessel 48 and the level sensor 51 to monitor the flow rate of biosolid particles through the vessel. With these sensor outputs and knowing the volume of the vessel 48, the controller calculates the actual residence time of biosolid particles in the vessel. For example, the actual residence time of biosolids is calculated by determining the time needed to file the vessel to the biosolid level in the tank at the current rate at which biosolids flow into or out of the vessel.

The temperature of the dried biosolids particles in the vessel 49 is monitored using one or more temperature sensors 64. These temperature sensors may be on the inside wall surface of the vessel 49 or mounted on a probe extending at least partially into the flow of biosolids in the vessel. The temperature signals from the sensors 64 are monitored and recorded by the control system 62. Using the temperature signals the control system can determine the temperature of the biosolids in the vessel.

The control system 62 is programmed with an executable software program that determines an appropriate temperature and residence time of biosolid particles in the vessel 48. The determination of a desired temperature or residence time for the biosolids may be based on an algorithm that establishes a relationship between residence time and temperature. The relationship may be, for example, the “time-temperature relationships” reported in Table 5-3 of the publication EPA/832/R-93/003 (Chapter 5, p. 112). An example of a time-temperature relationship is:

$$D \geq \frac{131 \text{,} 700 \text{,} 000}{{10}^{0.14 t}}$$

In this relationship, “D” represents the days of treatment of the biosolid needed to comply with the Part 503 Rule, and “t” represents the temperature in degrees Celsius of the treatment.

Using an algorithm to determine a desired residence time or temperature in the vessel 48, the control system 62 may regulate the flow rate of the biosolid particles through the vessel 48 by adjusting the rotational rate of discharge valve 60. This valve 60 has a known volumetric throughput based on speed of its rotation. By adjusting the rotational speed of the valve, such as by adjusting the speed of a valve drive motor, the controller 62 can reliably adjust the flow rate of biosolid particulates through the vessel 49. Similarly, the control system 62 may adjust the router 50 to regulate the flow rate of biosolids to the vessel 48.

To control the temperature of biosolid particles in the vessel 48, a hot fluid, e.g., gas, is percolated through the biosolid particles in the vessel. The hot gas may be a portion of the drum dryer exhaust gas taken from line 30 and supplied to the vessel through line 68. The gas may be introduced into a lower level of the vessel 48 through an array of gas discharge nozzles 53 in the bottom surface of the vessel. As the hot gases percolate through the biosolid particles in the vessel, heat from the gases is transferred to the particles. The gases are exhausted 73 from the top of the vessel and returned to the dryer exhaust line 30 through line 70. Control vents or fans 72 regulate the flow of the hot fluid to and from the vessel 48 in lines 68 and 70. The control system 62 may regulate the flow rate of the hot fluid to or from the vessel 49 by adjusting (A) the vents or fans 72. The controller may adjust the flow of hot fluid, e.g., gases, that percolate through the vessel 48 to ensure that the temperature of the particles in the vessel is no cooler than a minimum particle temperature, determined from the algorithm for the time-temperature relationship. The controller may increase the rate of hot fluid introduced into the vessel if the actual temperature of biosolids in the vessel, as determined from temperature sensors 64 is at or below a determined minimum temperature.

By maintaining the biosolid particles in the vessel 48 at least at a desired temperature and for at least a desired residence time, pathogens in the particles can be reduced to acceptable levels and compliance can be ensured with government regulations regarding treatment of sewage wastes for pathogens, e.g., EPA Part 503 Rule. The pathogen treatment vessel 48 provides a system for automatically treating pathogens in dried biosolids, e.g., dried sewage sludge, and the treatment is performed promptly after the biosolids are dried and reduced to particulate form. In particular, the dried biosolids are conveyed continuously and directly to the treatment vessel 48 without any intermediate temporary storage of the dried material.

From the discharge of the pathogen treatment vessel 48, the dried biosolids are temporarily stored in a silo 74 that may include cooling coils 76 that circulate a cooling fluid, e.g., water, through the silo. The silo 74 allows the biosolids to cool, e.g., to ambient temperature. A control valve 80 at the discharge of the silo 74 regulates the flow of dried biosolids to an elevator conveyor 82, which in combination with a screw conveyor 84 feeds the biosolids to a storage bin 86. The biosolid particles, treated to reduce pathogens, are stored in the bin 86 until the particles are sold, distributed as fertilizer on fields, e.g., grass fields, or otherwise applied.

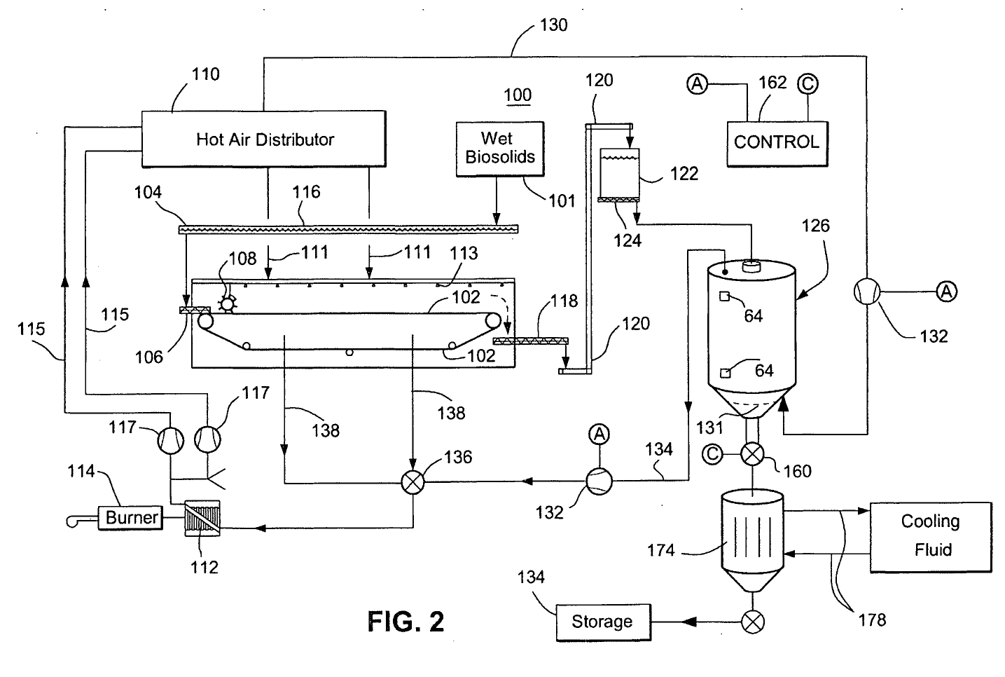

FIG. 2 is a schematic diagram of a biosolids drying system 100 having a particle filtering belt dryer 102, in which biosolids 101 to be dried are fed to an open screw conveyor 116. From the discharge 104 of the screw conveyor 116, distribution screw 106 moves the partially dried biosolids to a belt conveyor 102. The biosolids are spread out on the belt by a calibrating roll 108.

Hot air passing through the belt dryer 102 dries the wet biosolids on the filter belt. Air conduits 111 direct air from a hot air distributor 110 to air nozzles 113 above the belt conveyor 102. After passing through the belt dryer, the air is collected and is reheated in a heat exchanger 112. The air in the heat exchanger may be heated directly or indirectly by a burner 114. The heated air is directed to by conduits 115 the air distributor to be circulated through a screen conveyor 116 and the belt conveyor 102. Fans 117 may pump the hot air to the air distributor 110. From the air distributor, the hot air passes through the belt dryer 102 to dry the wet biosolids on the belt. Before reaching the belt, the hot air may also pass trough an open trough extended screw conveyor 116 that feeds the wet biosolids to the inlet to the belt dryer 102. The wet biosolids are preheated and partially dried in the screw conveyor 116 and are subsequently dried on the belt dryer 102. The dried biosolids fall of the end of the belt dryer 102 and into a screw conveyor 118.

The dried biosolid particulates material, for example biosolid particulates, is brought by the conveying screw 118 and a conveying elevator 120 to a backfeed silo 122 from where the dried biosolid particles are conveyed by a screw conveyor 124 to a pathogen treatment vessel 126. The pathogen treatment vessel 126 and cooling system 74 operate in a similar manner as dos the pathogen treatment vessel 48 and cooling system 74 shown in FIG. 1. The above description of those systems shown in FIG. 1 is fully applicable to the pathogen treatment vessel 126 and cooling system 74 shown in FIG. 2.

The dried biosolid particulate material is retained in the pathogen treatment vessel 126 for at least a prescribed residence time. The rate of flow of the biogen particulates through the vessel 126 is regulated by a rotary valve 60 that is controlled (C) by a control system 162. The controller reads temperature sensors 64 that monitor the temperature of the biosoild particulate material in the vessel 126. The control system executes an algorithm to determine appropriate minimum temperatures and residence times of biosolids in the pathogen vessel. To ensure that the biosolids are at or above the determined minimum temperature and residence times, the control system may adjust (see control signals A and C) the flow rate of biosolids through the vessel, e.g., by regulating valve 60, or adjust the temperature of the biosolids in the vessel.

Temperature control of the biosolids in the vessel may be provided by hot gases that percolate through the vessel 126. Gases from the hot air duct or distributor 110 may be fed from a gas line 130 to an array of gas nozzles 131 at a bottom inlet of the vessel 126. A fan or other air flow control device 132 may pump the air into the vessel. The fan controlled by the control system 162 to regulate the amount of hot gas added to the vessel 126. The hot air percolating through the biosolid particles in the vessel 126 heats the biosolid particles in the vessel. The amount of hot gases percolating through the vessel is determined by the controller which uses the temperature signals (B) from the temperature sensors 64 as a feed back signal to control the temperature of the particles in the vessel.

Hot gases are removed form the top of the vessel 126 into gas conduit 134. The hot gases from the vessel 126 may be mixed 136 with hot gases 138 from the belt dryer 102. The mixture of gases is reheated in the heat exchanger 112 and recirculated to the air distribution duct or distributor 110.

Biosolid particles are discharged from the vessel 126 after residing in the vessel for at least the prescribed residence time and heated in the vessel to at least the desired minimum temperature. The biosolid particles are discharged at a rate determined by valve 160. The discharged particles have a relatively low level of pathogens because their residence time and temperature in the vessel 126 minimized pathogens. From the vessel 126, the particles are cooled in a cooling vessel 174 which may be cooled with a cooling water 178. The cool biosolid particles are stored in a storage silo 134 until needed for fertilizer or other purposes.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A method for treating biosolid material having pathogens, the method comprising:
introducing dried biosolid material into a pathogen treatment vessel;
monitoring an actual temperature of the dried biosolid material in the treatment vessel;
determining a desired residence time of the biosolid material in the treatment vessel;
determining a desired minimum temperature of the dried biosolid material in the treatment vessel, wherein the minimum temperature is based on the desired determined residence time of the biosolid material in the treatment vessel;
passing the dried biosolid material through the treatment vessel such that the actual residence time of the biosolid material in the treatment vessel is at least as long as the determined residence time;
applying heat from a hot fluid to the dried biosolid material in the treatment vessel, if the actual temperature of the biosolid material is lower than the minimum temperature, and
reducing pathogens in the biosolid material by maintaining the biosolid material in the treatment vessel for at least the determined residence time while the actual temperature is at least as hot as the minimum temperature.

2. The method as in claim 1 wherein the dried biosolid material is a dried particulate material.

3. The method as in claim 1 wherein the dried biosolid material is dried sewage sludge.

4. The method as in claim 1 wherein the dried biosolid material is discharged from a drum dryer or belt dryer and flows to the pathogen treatment vessel.

5. The method as in claim 4 wherein the dried biosolid material flows continuously from the dryer to the treatment vessel without intermediate storage of the material.

6. The method as in claim 1 wherein the dried biosolid material is introduced at a continuous rate into the treatment vessel.

7. The method as in claim 1 wherein the hot fluid is a hot gas and the hot gas is percolated through the dried biosolids in the treatment vessel.

8. The method as in claim 1 including maintaining a substantially constant elevation of an upper level of the dried biosolids in the treatment vessel.

9. A method for treating biosolid material having pathogens, the method comprising:
drying the biosolid material in a dryer;
continuously conveying the dried biosolid material to a pathogen treatment vessel;
determining a desired residence time of the biosolid material in the treatment vessel;
monitoring an actual temperature of the dried biosolid material in the treatment vessel;
determining a desired minimum temperature of the dried biosolid material in the treatment vessel;
regulating a flow rate of the dried biosolid material through the treatment vessel such that the actual residence time of the biosolid material in the treatment vessel is at least as long as the determined residence time;
applying heat from a hot fluid to the dried biosolid material in the treatment vessel, and
reducing pathogens in the biosolid material by maintaining the biosolid material in the treatment vessel for at least the determined residence time while the actual temperature is at least as hot as the minimum temperature.

10. The method as in claim 9 wherein the dried biosolid material is at least one of a dried particulate material or dried sewage sludge.

11. The method as in claim 9 wherein the dried biosolid material flows continuously from the dryer to the treatment vessel without intermediate storage of the material.

12. The method as in claim 9 wherein the dried biosolid material is introduced at a continuous rate to the treatment vessel.

13. The method as in claim 9 wherein the hot fluid is a hot gas and the hot gas is percolated through the dried biosolids in the treatment vessel.

14. The method as in claim 9 including maintaining a substantially constant elevation of an upper level of the dried biosolids in the treatment vessel.

15. A system for treating biosolid material having pathogens comprising:
a dryer drying the biosolid material;
a conveyor continuously conveying the dried biosolid material to a pathogen treatment vessel;
the treatment vessel including an inlet to receive a continuous flow of the dried biosolid material and an outlet to discharge the continuous flow of the dried biosolid material;
a hot fluid inlet to and outlet from the treatment vessel;
a temperature sensor monitoring a temperature of the dried biosolid material in the treatment vessel;
a computer controller executing a program to determine a desired residence time of the biosolid material in the treatment vessel based on a desired minimum temperature of the dried biosolid material in the treatment vessel;
a dried solids flow regulator coupled to the inlet or outlet and regulating the continuous flow of the dried biosolid material through the treatment vessel, wherein the flow regulator is automatically adjusted by the computer controller to achieve a residence time of the biosolid material in the treatment vessel which is no shorter than the desired residence time, and
a hot fluid flow regulator regulating a flow of the hot fluid through the treatment vessel such that the monitored temperature of the biosolid material in the treatment vessel is no cooler than the desired minimum temperature.

16. The apparatus as in claim 15 wherein the computer controller further executes a program to determine the desired minimum temperature.

17. The apparatus as in claim 15 wherein the conveyor provides a continuously conveyor for moving the dried biosolid material to the treatment vessel without temporary storage of the biosolid material.

18. The apparatus as in claim 15 wherein the conveyor includes a router directing the dried biosolids material on the conveyor to the treatment vessel or to another conveying system.

19. The apparatus as in claim 15 wherein the hot fluid inlet and outlet are a hot gas inlet and a hot gas outlet.

20. The apparatus as in claim 19 wherein the hot fluid flow regulator is a fan or vent for the hot gas.
